(19) United States
(12) Patent Application Publication
(10) Pub. No.: US 2010/0087873 A1
(43) Pub. Date: Apr. 8, 2010

(54) Surgical Connectors for Attaching an Elongated Member to a Bone

(75) Inventors: William Barry Null, Olive Branch, MS (US); Marc Thomas Paul, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDICS, INC., Warsaw, IN (US)

(21) Appl. No.: 12/245,843

(22) Filed: Oct. 6, 2008

(51) Int. Cl. A61B 17/04

(52) U.S. Cl. 606/306; 606/305

(57) ABSTRACT

The present application is directed to connectors for attaching an elongated member to a bone. The connectors may include a receiver that is attached to an anchor. The receiver may include a base and outwardly-extending arms that form a channel to receive the elongated member. The base may include an opening that extends into a receptacle. The opening may include a first section in an inferior side of the receiver, and a cut-out in a lateral side. During attachment, the anchor may be positioned relative to the receiver such that a head of the anchor may be inserted into the first section and a shaft of the anchor may be inserted into a second section. After insertion, the anchor is rotated to move the shaft out of the cut-out while the head remains in the receptacle. A wedge is then attached to the receiver over the cut-out to prevent the anchor from escaping.

### BACKGROUND

The present application is directed to connectors for attaching an elongated member to a bone, and more particularly, to a connector with a receiver having a cut-out section for inserting an anchor into the receiver.

Elongated members are used in various surgical applications, such as treatment of fractures. Another context is in the surgical treatment of spinal disorders such as degenerative disc disease, disc herniations, scoliosis or other curvature abnormalities, and fractures. Treatment of these spinal disorders may use different types of surgical treatments. In some cases, spinal fusion is indicated to inhibit relative motion between vertebral members. In other cases, dynamic implants are used to preserve motion between vertebral members. For either type of surgical treatment, elongated members may be attached to the exterior of two or more vertebral members, whether it is at a posterior, anterior, or lateral side of the vertebral members. In other embodiments, elongated members are attached to the vertebral members without the use of dynamic implants or spinal fusion.

Elongated members may provide a stable, rigid column that encourages bones to fuse after spinal-fusion surgery. Further, the elongated members may redirect stresses over a wider area away from a damaged or defective region. Also, rigid elongated members may restore the spine to its proper alignment. In some cases, flexible elongated members may be appropriate. Flexible elongated members may provide other advantages, such as increasing loading on interbody constructs, decreasing stress transfer to adjacent vertebral members while bone-graft healing takes place, and generally balancing strength with flexibility.

The elongated members are secured to one or more vertebra through connectors. The connectors include a receiver that receives the elongated member, and an anchor to anchor into the vertebra. The receiver and anchor should be constructed in a manner to allow for these elements to be connected together in an effective manner.

### SUMMARY

The present application is directed to connectors for attaching an elongated member to a bone. The connectors may include a receiver that is attached to an anchor. The receiver may include a base and outwardly-extending arms that form a channel to receive the elongated member. The base may include an opening that extends into a receptacle. The opening may include a first section in an inferior side of the receiver, and a cut-out in a lateral side. During attachment, the anchor may be positioned relative to the receiver such that a head of the anchor may be inserted into the first section and a shaft of the anchor may be inserted into a second section. After insertion, the anchor is rotated to move the shaft out of the cut-out while the head remains in the receptacle. A wedge is then attached to the receiver over the cut-out to prevent the anchor from escaping.

The various aspects of the various embodiments may be used alone or in any combination, as is desired.

### BRIEF DESCRIPTION OF THE DRAWINGS

FIG. 1 is a perspective view of a connector according to one embodiment.

FIG. 2 is an exploded perspective view of a connector according to one embodiment.

FIG. 3 is a perspective view of a receiver according to one embodiment.

FIG. 4 is a sectional view of a receiver and a wedge according to one embodiment.

FIG. 5 a bottom view of an anchor extending outward from a receiver with an attached wedge according to one embodiment.

FIG. 6 side view of a receiver and wedge with an attached anchor according to one embodiment.

FIG. 7 is a perspective view of a first side of a wedge according to one embodiment.

FIG. 8 is a perspective view of a second side of a wedge according to one embodiment.

FIG. 9 is a side view of a receiver with a crown and anchor positioned with their axes perpendicular to one another according to one embodiment.

FIG. 10 is a sectional view of the receiver and anchor cut along lines 10-10 of FIG. 9.

FIG. 11 is a sectional view of a connector with axes of the receiver and anchor aligned according to one embodiment.

### DETAILED DESCRIPTION

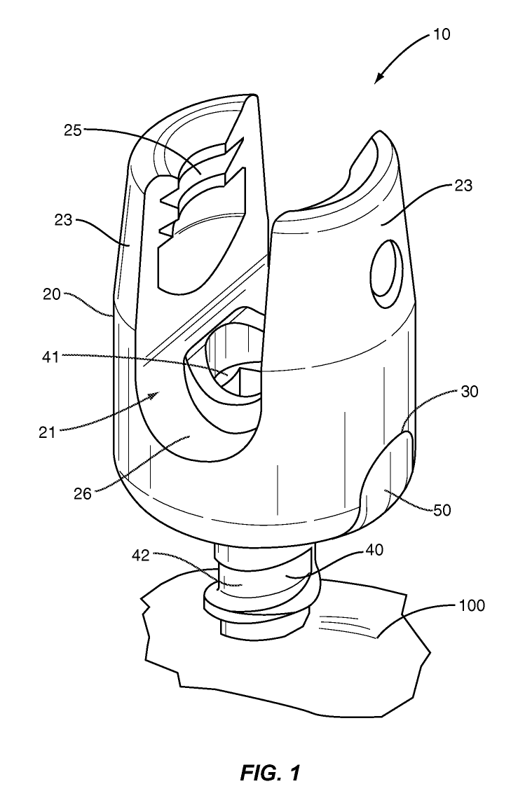

The present application is directed to connectors for attaching an elongated member to a bone. FIG. 1 illustrates one embodiment of a connector 10 that includes a receiver 20 and an anchor 40. The anchor includes a head 41 and shaft 42. The receiver 20 includes a channel 21 formed between a pair of arms 23 sized to receive the elongated member. A fastener (not illustrated in FIG. 1) attaches to the arms 23 to prevent escape of the elongated member from the channel 21. Receiver 20 also includes a base 22 with an interior sized to receive the head 41. A cut-out 30 in the base 22 extends into the interior. The cut-out 30 is sized to receive the shaft 42 during insertion of the head 41 into the interior. Once the anchor 20 is inserted in the interior, a wedge 50 is attached to the receiver 20 and positioned in the cut-out 30 to prevent escape of the head 41 from the interior and maintain the anchor 40 attached to the receiver 20.

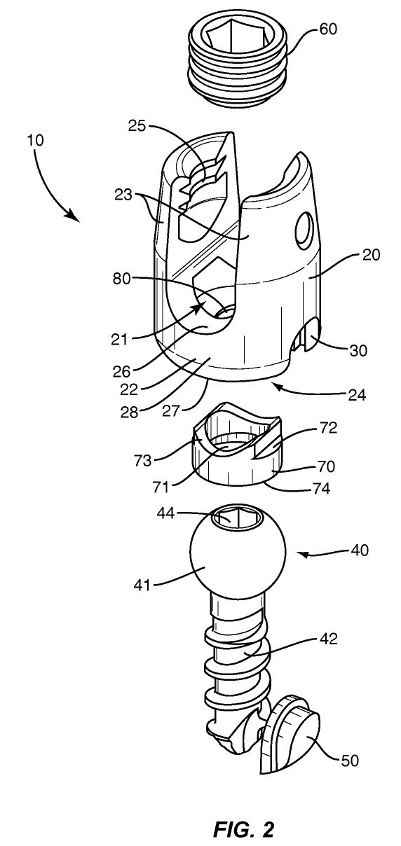

FIG. 2 is an exploded view of a connector 10. The receiver 20 includes a base 22 with a superior side with outwardly-extending arms 22 that are spaced apart to form a channel 21. A fastener 60 is sized to engage with the arms 22 to prevent escape of the elongated member from the channel 21. The base 23 also includes an interior receptacle 80 sized to receive a head 41 of the anchor 40. An opening 24 in the inferior side of the base 23 leads into the receptacle 80. A cut-out 30 extends through a sidewall of the base 23 and is in communication with the opening 24. The combined opening 24 and cut-out 30 are sized to allow the head 41 to be inserted into the receptacle 80. A wedge 50 attaches to the receiver 20 and extends over the cut-out 30 to prevent escape of the head 41 once it has been inserted in the receptacle 80. A crown 70 may be positioned in the receptacle 80 between the head 41 and the channel 21.

The receiver 20 may include a generally cylindrical shape with a curved exterior surface and a pair of opposing arms 23 that extend outward from a base 22. The arms 23 may include threaded sections 25 that engage with the fastener 60. The threaded sections 25 may be positioned on an interior surface of the arms 23 as illustrated in FIGS. 1 and 2, or may be positioned on an exterior surface. The arms 23 may be substantially the same, or may include different shapes and/or sizes.

The base 22 includes a superior side 26, an inferior side 27, and a sidewall 28 therebetween. A receptacle 80 is positioned within the base 22 and is sized to receive the head 41 of the anchor 40. The superior side 26 may form a lower extent of the channel 21 and be curved to match the shape of the elongated member. The inferior side 27 may include a rounded shape to facilitate movement relative to the anchor 40.

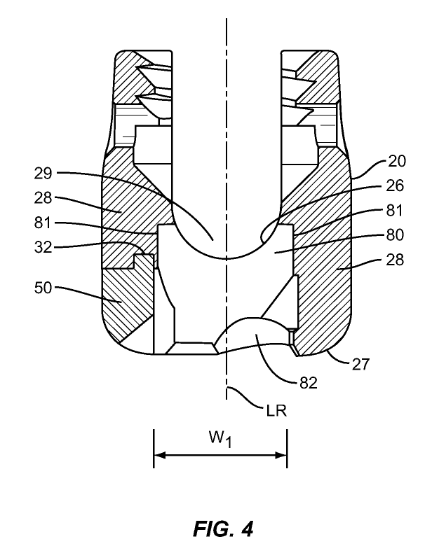

The receptacle 80 is positioned within the base 22 and includes a width W1. As illustrated in FIG. 4, the receptacle 80 is centered on a longitudinal axis LR of the receiver 20. Notches 81 formed in the interior of the receiver 20 may be size to position the crown 70 in the receptacle 80. An opening 29 in the superior side 26 may lead between the receptacle 80 and the channel 21.

The opening 24 extends through the inferior side 27 of the receiver 20 and leads into the receptacle 80. The opening 24 may also be centered on the longitudinal axis LR of the receiver 20. The opening 24 includes a smaller width than the receptacle 80.

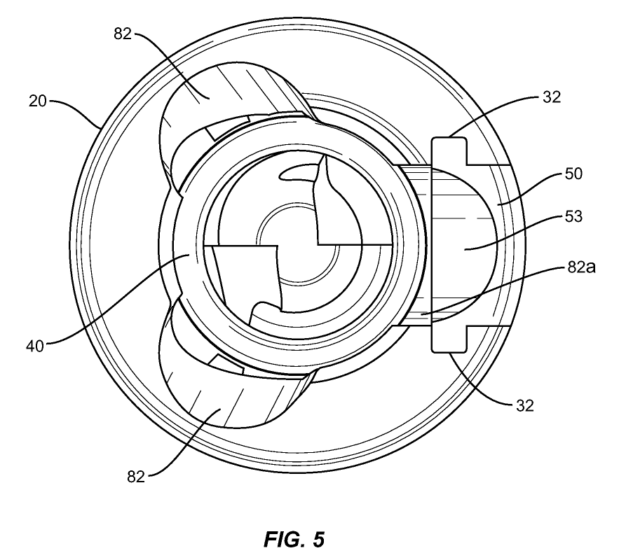

One or more recesses 82 are positioned on the inferior side 27 of the receiver 20 to allow increased angulation of the anchor 40 relative to the receiver 20 once the connector 10 is assembled. Each recess 82 includes an angled surface that angles outward away from the longitudinal axis LR a greater amount than the adjacent sections of the inferior side 27. The recesses 82 may be evenly spaced around the opening 24, such as the embodiment of FIG. 5 that includes the recesses 82 centered about 120 degrees apart.

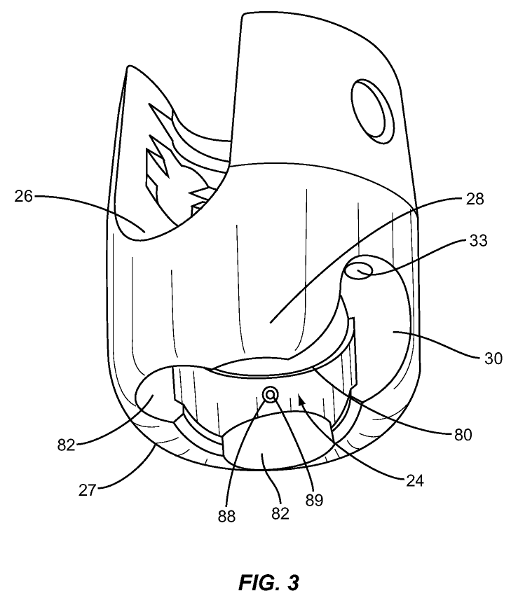

The cut-out 30 extends through the sidewall 28 of the receiver 20 and into the receptacle 80. The cut-out 30 is in communication with the opening 24 such that the cut-out 30 and opening 24 together form a single, continuous opening that leads into the receptacle 80. The cut-out 30 may include a superior side 31 and opposing lateral edges. The cut-out 30 includes a width that is greater than a width of the anchor shaft 42. The width of the cut-out 30 may also be smaller than a width of the anchor head 41. The superior edge 31 may include a curved shape that matches a shape of the shaft 42. In one embodiment, the superior edge 31 is positioned in closer proximity to the inferior side 27 of the receiver 20 than the superior side 26 of the receiver 20. Slots 32 may extend outward from the cut-out 30 and into the interior of the sidewall 28. The slots 32 may extend along one or more of the superior side 31 and lateral sides as illustrated in FIGS. 4 and 5. As illustrated in FIG. 3, a cavity 33 may extend into the sidewall 28 beyond the superior side 31.

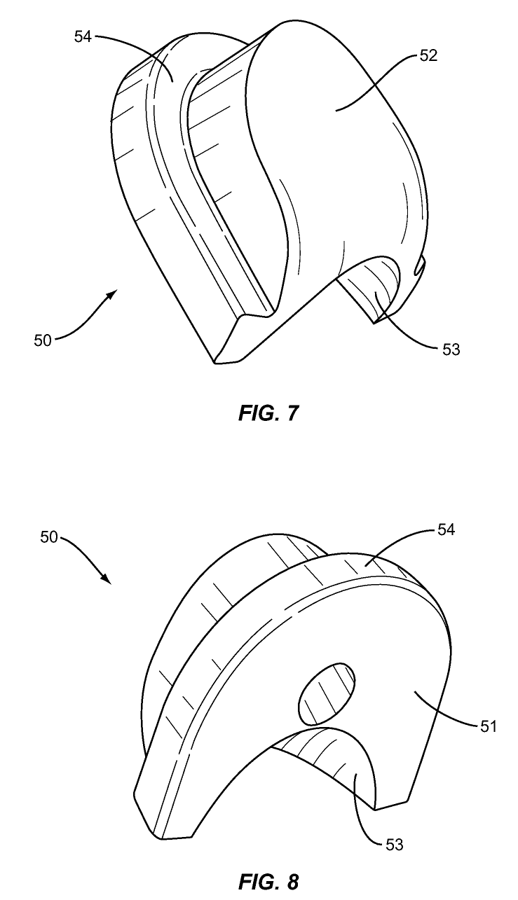

The wedge 50 fits in the cut-out 30 and prevents escape of the anchor head 41. The wedge 50 may completely fill the cut-out 30, or just a partial section of the cut-out 30. As best illustrated in FIGS. 7 and 8, wedge 50 includes an interior surface 51 that faces into the receptacle 80, and an exterior surface 52. When the wedge 50 is attached to the receiver 20, the interior surface 51 aligns with the sidewall interior and the wedge 50 may not extend into the receptacle 80. Also when the wedge 50 is attached, the exterior surface 52 aligns with and may be flush with the exterior surface of the receiver 20. The exterior surface 52 may be curved to match the curvature of the exterior of the receiver 20. A flange 54 extends around a portion of the periphery and is sized to seat within slots 32 of the cut-out 30 to facilitate mounting. Further, edges of the wedge 50 may contact against the superior edge 31 and lateral edges of the cut-out 30. The wedge 50 may be attached to the receiver 20 in various manners, including but not limited to spot welding, adhesives, mechanical fasteners, and combinations thereof.

Wedge 50 may further include a recess 53 that allows for additional angulation of the anchor 20. As best illustrated in FIG. 5, the recess 53 may be evenly spaced with the other recesses 82 in the receiver 20. In one embodiment, the cut-out 30 includes a portion of a recess 82a. The recess 53 in the wedge 50 aligns with the partial recess 82a to form a complete recess that is the same size and shape as the recesses in the receiver 20.

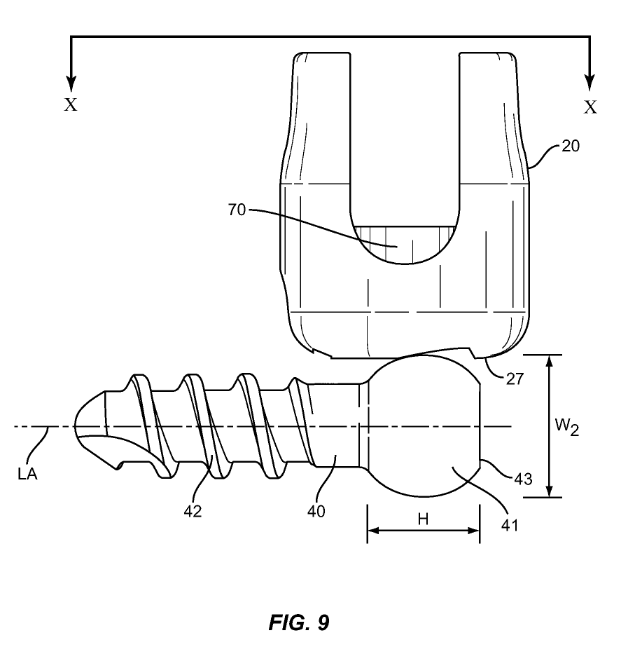

The anchor 40 includes a head 41 and a shaft 42. The head 41 includes a spherical shape with a flat top 43 that is positioned opposite from the shaft 42. As illustrated in FIG. 9, the head includes a width W2 measured between opposing spherical sides of the head 41 and perpendicular to a longitudinal axis LA of the anchor 40. The width W2 is less than the width W1 of the receptacle 80 and greater than a width of the opening 24. The shaft 42 is straight and extends outward from the head 41. The shaft 42 may also include threads to facilitate engagement with the bone. The width of the shaft 42 measured perpendicular to the longitudinal axis LA is less than a width of the cut-out 30. Attachment structure 44 may be positioned in the head 41 and configured to receive a drive tool for inserting the anchor 40 into the bone. Attachment structure 44 may include a polygonal recess, or other like structure to engage with the drive tool.

The fastener 60 secures the elongated member in the channel 21 of the receiver 20. FIG. 2 illustrates on embodiment with the fastener including threads 61 on an exterior surface that engage with threads 25 on the interior of the arms 23. In another embodiment (not illustrated), fastener 60 includes a central opening sized to extend around the arms 23. In this embodiment, threads are positioned around the central opening to engage with threads on the exterior of the arms 23.

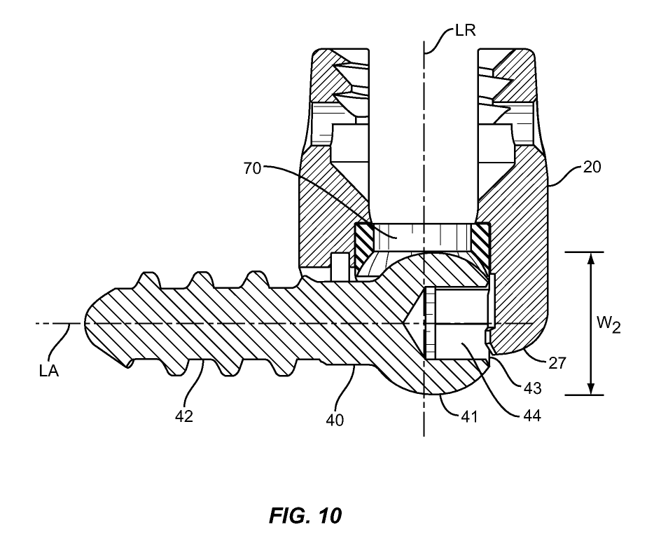

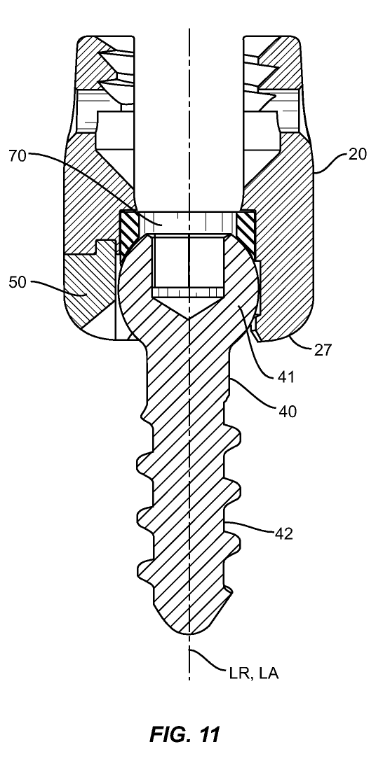

Crown 70 is configured to be positioned in the receiver 20 and includes an annular shape with a central opening 71. When positioned in the receiver 20, the opening 71 aligns with the second opening 29 to provide access to the attachment structure 44 of the anchor 40. Crown 70 includes a superior surface 73 and an inferior surface 74. The surfaces 73, 74 may be flat, or may include one or more undulations. Crown 70 may include a shoulder 74 that corresponds with the notches 81 in the receptacle 80 as illustrated in FIGS. 10 and 11. The crown 70 may be constructed from a softer material than the head 41 which allows the crown and head 41 to interdigitate when the crown 70 is forced onto the head 41 by the elongated member from tightening the fastener 60.

One or more biasing members 89 may extend into the receptacle 80 as illustrated in FIG. 3. The biasing members 89 contact against the head 41 of the anchor 40 and provide a force against the head 41 to prevent or limit motion of the anchor 40 relative to the receiver 20. Biasing members 89 may be formed by a coil spring, washer, silicone plug, and others. In embodiments with multiple biasing members 89, the biasing members 89 may each be the same or may be different.

The biasing members 89 are each positioned in a cavity 88 formed in an interior wall of the receiver 20. The cavities 88 and biasing members 89 may be positioned at different locations around the receiver 20. In one embodiment, the cavities 88 and biasing members 89 are positioned opposite from a non-recessed section of the receiver 20. Also, the cavities 88 and biasing members 89 may be positioned above one or more of the recesses 82. The cavities 88 are positioned for the biasing members 89 to contact against the rounded section of the head 41, and not to contact against the flat top 43. Contact against the flat top 43 may prevent the anchor 40 from rotating back to a centered position with the longitudinal axis LA of the anchor 40 aligned with a longitudinal axis LR of the receiver 20.

The number of cavities 88 and biasing members 89 may vary. In one embodiment, two cavities and biasing members 89 are positioned about 120 degrees apart. In another embodiment, a single cavity and biasing member 89 is positioned in the receiver 20. One or more cavities and biasing members 89 may also be positioned on the wedge 50. FIG. 8 includes an embodiment with a cavity positioned in the wedge 50.

FIGS. 9, 10, and 11 illustrate one method of attaching the anchor 40 and receiver 20. Prior to attachment, the cut-out 30 is not attached to the receiver 20. The anchor 40 is positioned with the longitudinal axis LA at a non-parallel angle with the longitudinal axis LR of the receiver 20 as illustrated in FIGS. 9 and 10. The anchor 40 may be perpendicular to the receiver 20, or at a lesser non-parallel angle (i.e., less than 90 degrees) that allows for the head 41 to be inserted through the opening 24 and into the receptacle 80 with the shaft 42 being inserted into the cut-out 30. In one embodiment as illustrated in FIG. 9, the height H of the head 41 measured between the flat edge 43 and the base of the head 41 is smaller than the width of the opening 24 and the width W1 of the receptacle 80. FIG. 10 also illustrates that the width W2 of the head 41 may be greater than a height of the receptacle 80 such that a portion of the head 41 extends beyond the inferior end 27 when the axes LA, LR are non-parallel.

Once the head 41 is in the receptacle 80, the anchor 40 is rotated relative to the receiver 20 such that the axes LA, LR are in closer alignment. In one embodiment as illustrated in FIG. 11, the axes LA, LR may be in total alignment. The relative movement between the anchor 40 and receiver 20 causes the head 41 to pivot in the receptacle 80 as the shaft 42 moves out of the cut-out 30. The head 41 is able to pivot in the receptacle 80 because the width W2 of the head 41 is greater than the receptacle width W1.

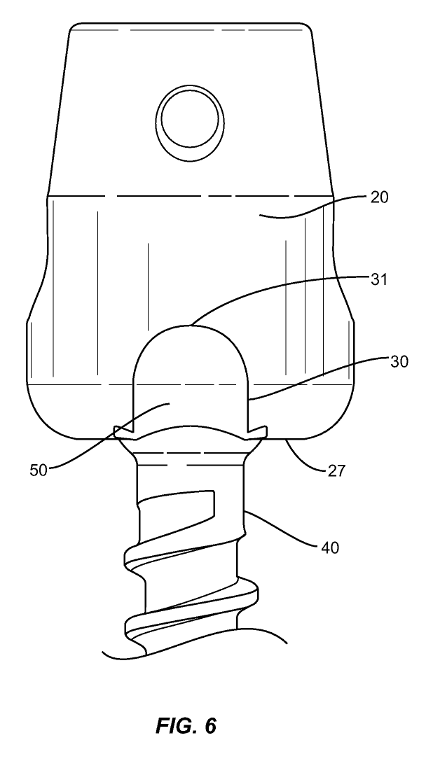

To prevent the anchor 40 from escaping from the receiver 20, the wedge 50 is attached to the receiver 20. In one embodiment, the wedge 50 is placed below the receiver 20 and inserted through the inferior side 27 and moved upwardly into the cut-out 30. This direction of insertion seats the flange 54 of the wedge 50. In one embodiment as illustrated in FIG. 6, the wedge 50 extends completely across the cut-out 30. The outer edges of the exterior section of the wedge 50 contact against the edges of the cut-out 30. The interior section mates with corresponding structure of the receiver 20. The exterior surface 52 may be flush with the exterior surface of the receiver 20. Further, the exterior surface 52 may match the exterior surface of the receiver 20 giving the visual indication that the receiver 20 is a single, continuous piece. As illustrated in FIG. 5, the exterior surface 52 may include a curved shape that matches the curved shape of the receiver 20. The inferior edge of the wedge 50 may also match the inwardly-curving (i.e., curving towards the longitudinal axis LR) shape of the receiver 20. Further, the interior surface 51 may also align with and match the shape of the inner sidewall of the receiver 20. Further, the wedge 50 may be sized and shape such that the interior surface 51 does not extend into the receptacle 80.

Once positioned relative to the cut-out 30, the wedge 50 is attached to the receiver 20. The attachment may be preformed by spot welding, adhesives, mechanical fasteners, and various other methods. Once attached, the wedge 50 prevents the head 41 from escaping out of the receptacle 80. The wedge 50 is sized to reduce the remaining size of the opening 24 to be less than that of the head 41. The remaining opening 24 may be symmetrical or non-symmetrical. After the wedge 50 is attached to the receiver 20, the anchor 40 may still be movable relative to the receiver 20 to allow adjustment of the angular position of the anchor 40.

The connector 10 may be used in a variety of contexts to attach an elongated member to a bone. Examples include but are not limited to attaching a vertebral rod to a vertebra and attaching a rod to a fractured femur.

The receiver 20 and wedge 50 may be formed from a variety of materials including but not limited to titanium, stainless steel, carbon fiber, and polyetheretherketone (PEEK). The receiver 20 and wedge 50 may be formed from the same or different materials.

Spatially relative terms such as “inferior”, “superior”, “lower”, “over”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The wedge 50 may be smaller than the size of the cut-out such that it does not extend completely across the cut-out 30. The present embodiments are, therefore, to be considered in all respects as illustrative and not restrictive, and all changes coming within the meaning and equivalency range of the appended claims are intended to be embraced therein.

What is claimed is:

1. A connector to attach an elongated rod to a bone comprising:
a receiver with a base and opposing arms that extend outward from a first side of the base to form a channel to receive the elongated rod, the receiver including a longitudinal axis;
a receptacle in the base with a receptacle width measured perpendicular to the longitudinal axis of the receiver;
an anchor including a head positioned within the receiver and a shaft, the head including a head width measured perpendicular to a longitudinal axis of the anchor and a head height measured along the longitudinal axis, the head width being less than the receptacle width and the head height being less than the receptacle width, the shaft including a shaft width measured perpendicular to the longitudinal axis of the anchor;
an opening in a second side of the base opposite from the channel that leads into the receptacle, the opening including an opening width that is less than the receptacle width and less than the head width;
a cut-out in a lateral side of the receiver between the first and second sides of the base, the cut-out extending into the receptacle and the opening, the cut-out including a cut-out width that is greater than the shaft width and less than the head width; and
a wedge configured to attach to the receiver and fit in the cut-out, the wedge including an inner surface that faces towards the receptacle and an outer surface that faces away from the receptacle, the outer surface being aligned with an exterior surface of the receiver when the wedge is attached to the receiver.

2. The connector of claim 1, wherein the inner surface of the wedge includes a larger width than the outer surface.

3. The connector of claim 2, wherein the wedge includes a flange that extends around a section of the inner surface and fits within a slot formed in the receiver that is positioned along the cut-out.

4. The connector of claim 1, wherein the outer surface includes a curved shape that matches an exterior curvature of the receiver.

5. The connector of claim 1, wherein the second side of the base includes non-recessed sections and a plurality of recessed sections that each angle away from the longitudinal axis of the receiver a greater amount than the non-recessed sections.

6. The connector of claim 1, wherein the wedge includes a recess formed on an inferior side that aligns with the second side of the base when the wedge is attached to the receiver, the recess including a scalloped edge that angles away from the longitudinal axis of the receiver.

7. The connector of claim 1, wherein a superior edge of the cut-out is positioned in closer proximity to the second side of the base than the channel.

8. The connector of claim 1, wherein the head includes a flat surface positioned opposite from the shaft.

9. The connector of claim 1, further including at least one biasing member attached to the receiver and extending into the receptacle, the biasing member being aligned above a recess formed in the receptacle and opposite from a non-recessed section.

10. A connector to attach an elongated rod to a bone comprising:
a base with a superior side, inferior side, and a sidewall therebetween, the base including a longitudinal axis that extends through a center of the base;
a pair of opposing arms that extend outward from the superior side of the base and form a channel configured to receive the elongated rod;
a receptacle formed within an interior of the base;
an opening in the base that extends into the receptacle, the opening including a first section positioned along the inferior side and a second section in communication with the first section that extends along the sidewall, the second section including opposing lateral sides and a superior side;
an anchor including a head positioned in the receptacle and a shaft extending outward from the head, the head including a width that is greater than a width of the second section and the shaft including a width that is smaller than the width of the second section; and
a wedge sized to fit within the second section of the opening and prevent removal of the head from the base, the wedge sized to contact against the lateral sides and the superior side of the second section and including an inferior edge that aligns with the inferior side of the base when the wedge is attached to the base, the wedge further including an exterior surface that is flush with an exterior of the base when the wedge is attached to the base.

11. The connector of claim 10, wherein the first section of the opening is centered on the longitudinal axis of the base.

12. The connector of claim 10, wherein an interior surface of the wedge is wider than the exterior surface.

13. The connector of claim 10, wherein a height of the head measured along a longitudinal axis of the anchor is less than a width of the receptacle.

14. The connector of claim 10, wherein the inferior side of the base includes a non-recessed section and a first recessed section that angles away from the longitudinal axis of the base a greater amount than the non-recessed section, the inferior edge of the wedge further includes a second recessed section that aligns with the first recessed section to form a complete recess when the wedge is attached to the base.

15. The connector of claim 10, wherein the superior side of the second section of the opening is positioned in closer proximity to the inferior side of the base than the channel.

16. A method of assembling a connector that attaches an elongated rod to a bone, the method comprising:
positioning an anchor relative to a receiver with a longitudinal axis of the anchor being perpendicular to a longitudinal axis of the receiver;
inserting a head of the anchor into an opening in an inferior side of the receiver and a shaft of the anchor into a cut-out that extends into the inferior side of the receiver;
positioning the head in a receptacle in the receiver that is in communication with the opening while the shaft is in the cut-out;
rotating the anchor relative to the receiver and moving the shaft out of the cut-out while the head remains in the receptacle;
while the head and the shaft are out of the cut-out, attaching a wedge into the cut-out and preventing the head from being removed from the receptacle.

17. The method of claim 16, further comprising attaching the wedge such that exterior surfaces of the wedge and the receiver are flush.

18. The method of claim 16, further comprising positioning a flat top on the anchor away from the cut-out while inserting the head of the anchor into the opening.

19. The method of claim 16, further comprising attaching the wedge into the cut-out and completing the opening such that a width of the opening is less than a width of the head.

20. The method of claim 16, further comprising positioned a biasing member in a cavity in an inner wall of the receptacle and contacting the head of the anchor against the biasing member.

21. A method of assembling a connector that attaches an elongated rod to a bone, the method comprising:
positioning an anchor relative to a receiver with a longitudinal axis of the anchor out of alignment with a longitudinal axis of the receiver;
inserting a head of the anchor into a first section of an opening on an inferior side of the receiver and a shaft of the anchor into a second section of the opening on a sidewall of the receiver;
positioning the head into a receptacle in the receiver that is in communication with the opening while the shaft is in the second section;
pivoting the anchor relative to the receiver such that the longitudinal axes are in closer alignment and moving the shaft out of the second section while the head remains in the receptacle;
attaching a wedge to the receiver with the wedge extending into the second section and reducing a width of the opening to be smaller than a width of the head and preventing the head from being removed from the receptacle.

22. The method of claim 21, further comprising aligning a second section of a recess on the wedge with a first section of the recess on the inferior side of the receiver while attaching the wedge to the receiver.

23. The method of claim 21, further comprising contacting outer edges of the wedge against edges of the receiver that form the second section when attaching the wedge to the receiver.

24. The method of claim 21, wherein attaching the wedge to the receiver includes completely covering the second section of the opening.

25. The method of claim 21, further comprising adjusting an angular position of the anchor relative to the receiver after attaching the wedge to the receiver.
